(19) United States
(12) Patent Application Publication
(10) Pub. No.: US 2024/0139108 A1
(43) Pub. Date: May 2, 2024

(54) ORAL PHARMACEUTICAL COMPOSITION OF ARSENIC TRIOXIDE

(71) Applicant: INTAS PHARMACEUTICALS LTD., Gujarat Ahmedabad (IN)

(72) Inventors: Nirav Nitinkumar BHAVSAR, Tal.: Sanand Gujarat, Ahmedabad (IN); Sudhir Santosh IGHE, Tal.: Sanand Gujarat, Ahmedabad (IN); Ashutosh JAMLOKI, Tal.: Sanand Gujarat, Ahmedabad (IN); Ajeet SINGH, Tal.: Sanand Gujarat, Ahmedabad (IN); Ashish SEHGAL, Tal.: Sanand Gujarat, Ahmedabad (IN)

(21) Appl. No.: 18/547,260

(22) PCT Filed: Feb. 25, 2022
(86) PCT No.: PCT/IB2022/051662

§ 371 Date: Aug. 21, 2023

(30) Foreign Application Priority Data
Feb. 26, 2021 (IN) 202121008186

(51) Int. Cl. A61K 9/16; A61K 9/00; A61K 33/36

(52) U.S. Cl. CPC A61K 9/1694; A61K 9/0053; A61K 33/36

(57) ABSTRACT

The present invention provides oral pharmaceutical composition comprising Arsenic trioxide and one or more pharmaceutically acceptable excipients for use in the treatment of acute promyelocytic leukemia and other cancers like acute myeloid leukemia. Further, the present invention provides a process for the preparation of the said composition.

### RELATED APPLICATIONS

This application is related to Indian Provisional Application No. IN 202121008186 filed on 26 Feb. 2021 and is incorporated herein in its entirety.

### FIELD OF THE INVENTION

The present invention provides an oral pharmaceutical composition comprising Arsenic trioxide and one or more pharmaceutically acceptable excipients for use in the treatment of acute promyelocytic leukemia and other cancers like acute myeloid leukemia. Further, the present invention provides a process for the preparation of the said composition.

### BACKGROUND OF THE INVENTION

Arsenic trioxide is an inorganic compound, which is chemically Diarsenic trioxide. The empirical formula of Arsenic trioxide is As₂O₃ and it has the structural formula as shown below.

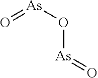

Arsenic trioxide is sold under the brand name as Trisenox® Injection. By injection into a vein, it is used for induction of remission and consolidation in patients with acute promyelocytic leukemia (APL). Trisenox® injection is stored at 20° C. to 25° C.; excursions permitted to 15° C. to 30° C.

The U.S. Pat. No. 6,723,351 discloses use of arsenic trioxide in acute promyelogenous leukemia (APL) in human administering 0.15 mg/kg arsenic trioxide once per day.

The U.S. Pat. No. 7,521,071 discloses method of treatment of hematological malignancies with the use of arsenic trioxide which is administered orally or intravenously. The orally administered arsenic trioxide produce lower peak plasma concentration with less prolongation of the QT interval and ventricular tachycardia than observed when the same amount of arsenic trioxide is administered intravenously. However, there is no guidance on how to prepare an oral formulation of arsenic trioxide in the description of the patent.

The CN Patent No. CN1189181 discloses oral slow-release granules of arsenic trioxide for acute promyelocytic leukemia (APL).

The CN Patent Nos. CN1269487 & CN103393719 discloses a process for the preparation of oral liquid of arsenic trioxide.

The US Patent No. U.S. Ser. No. 10/111,836 discloses an oral pharmaceutical formulation comprising a lyophilized arsenic trioxide and method for preparation of lyophilized arsenic trioxide.

However, a skilled person would understand that there are several limitations associated with lyophilized dosage forms, which includes the high manufacturing cost, complexity of equipment, increased handling and processing time. Therefore, lyophilization is not a preferred method for making oral pharmaceutical compositions.

Currently, there is no USFDA-approved oral arsenic trioxide available for treatment. Further, arsenic trioxide exhibits low solubility in water. Hence, there exists a need to develop an improved process for the preparation of oral arsenic trioxide composition, which is less tedious, more efficient, more economical and industrially feasible. The inventors of the present invention have developed oral pharmaceutical composition comprising Arsenic trioxide with improved and cost-effective process, and the said oral composition provides enhanced dissolution profile without the need for lyophilization.

### OBJECT OF THE INVENTION

The main object of the present invention is to provide an oral pharmaceutical composition comprising Arsenic trioxide and one or more pharmaceutically acceptable excipients.

Another object of the present invention is to provide an oral pharmaceutical composition comprising Arsenic trioxide and one or more pharmaceutically acceptable excipients, wherein the said composition is a capsule, a tablet or granules.

Another object of the present invention is to provide an oral pharmaceutical composition comprising Arsenic trioxide and one or more pharmaceutically acceptable excipients, wherein the amount of arsenic trioxide in the said composition is in the range from about 1 mg to 25 mg.

Another object of the present invention is to provide an oral pharmaceutical composition comprising Arsenic trioxide and one or more pharmaceutically acceptable excipients, wherein the said composition is not obtained by lyophilization.

Another object of the present invention is to provide an oral pharmaceutical composition comprising Arsenic trioxide and one of more pharmaceutically acceptable excipients, wherein the said composition is a capsule, a tablet or granules; wherein the said composition is not obtained by lyophilization, and wherein the composition comprises pharmaceutically acceptable excipients selected from diluent, binder, lubricant, glidant, surfactant, solvent, pH adjusting agents and optionally disintegrant.

Another object of the present invention is to provide a process for preparation of oral pharmaceutical composition comprising Arsenic trioxide and one or more pharmaceutically acceptable excipients in the form of granules, wherein the said granules are prepared by wet granulation process, spray drying process or by rotary evaporator.

Another object of the present invention is to provide a process for preparation of oral pharmaceutical composition comprising Arsenic trioxide and one or more pharmaceutically acceptable excipients in the form of granules, wherein the said granules are prepared by wet granulation process comprising of a fluid bed processor or a rapid mixer granulator.

Another object of the present invention is to provide a process for preparation of oral pharmaceutical composition comprising Arsenic trioxide and one or more pharmaceutically acceptable excipients in the form of granules, wherein the said granules are prepared by fluid bed processor or rapid mixer granulator; and the process for preparation includes the steps of:
1. Dissolving Arsenic Trioxide with pH adjusting agents in purified water,
2. Adding the binder solution and surfactant to the solution obtained in step 1,
3. Granulating the diluent with the solution obtained in step 2, to obtain the granules in a fluid bed processor or a rapid mixer granulator.
4. Mixing the lubricant and glidant with obtained granules.

Another object of the present invention is to provide a process for preparation of oral pharmaceutical composition comprising Arsenic trioxide and one or more pharmaceutically acceptable excipients in the form of granules, wherein the said granules are prepared by rotary evaporator; and the process for preparation includes the steps of:
1. Dissolving Arsenic Trioxide with pH adjusting agents in purified water till pH of about 8.0,
2. Preparing the solution of diluent in water separately to get the clear solution.
3. Adding the solution of step 2 to step 1 under stirring and adding glidant to the obtained solution under stirring.
4. Adding the above solution in the rotary evaporator and drying it to get the granules.
5. Mixing lubricant, surfactant and glidant with obtained granules.

Another object of the present invention is to provide an oral pharmaceutical composition comprising Arsenic trioxide and one or more pharmaceutically acceptable excipients, wherein the said composition is not obtained by lyophilization; and wherein at least 80% of arsenic trioxide in the composition is released within 45 minutes when measured by dissolution test in 900 ml of 0.1 N HCl as dissolution media having paddle with sinker at 100 RPM, or by dissolution test in 250 ml 0.1 N HCl as dissolution media having paddle with sinker at 50 RPM.

Another object of the present invention is to provide an oral pharmaceutical composition comprising Arsenic trioxide and one or more pharmaceutically acceptable excipients, wherein the said composition is not obtained by lyophilization; and wherein the said composition can be used for the treatment of acute promyelocytic leukemia and other cancers like acute myeloid leukemia.

### SUMMARY OF THE INVENTION

In a first embodiment, the present invention relates to an oral pharmaceutical composition comprising Arsenic trioxide and one or more pharmaceutically acceptable excipients, wherein the said composition is not obtained by lyophilization.

In another embodiment, the present invention relates to an oral pharmaceutical composition comprising Arsenic trioxide and one or more pharmaceutically acceptable excipients, wherein the said composition is in the form of a capsule, a tablet or granules.

In another embodiment, the present invention relates to an oral pharmaceutical composition comprising Arsenic trioxide and one or more pharmaceutically acceptable excipients, wherein the amount of arsenic trioxide in the said composition is from about 1 mg to 25 mg.

In another embodiment, the present invention relates to an oral pharmaceutical composition comprising Arsenic trioxide and one of more pharmaceutically acceptable excipients, wherein the said composition is in the form of a capsule, a tablet or granules; wherein the said composition is not obtained by lyophilization, and wherein the composition comprises pharmaceutically acceptable excipients selected from diluent, binder, lubricant, glidant, surfactant, solvent, pH adjusting agents and optionally disintegrant.

In another embodiment, the present invention relates to a process for preparation of oral pharmaceutical composition comprising Arsenic trioxide and one or more pharmaceutically acceptable excipients in the form of granules, wherein the said granules are prepared by wet granulation process, spray drying process or by rotary evaporator.

In another embodiment, the present invention relates to a process for preparation of oral pharmaceutical composition comprising Arsenic trioxide and one or more pharmaceutically acceptable excipients in the form of granules, wherein the said granules are prepared by wet granulation process comprising of fluid bed processor or rapid mixer granulator.

In another embodiment, the present invention relates to a provide process for preparation of oral pharmaceutical composition comprising Arsenic trioxide and one or more pharmaceutically acceptable excipients in the form of granules, wherein the said granules are prepared by fluid bed processor or rapid mixer granulator; and the process for preparation includes the steps of:
1. Dissolving Arsenic Trioxide with pH adjusting agents in purified water,
2. Adding the binder solution and surfactant to the solution obtained in step 1,
3. Granulating the diluent with the solution obtained in step 2, to obtain the granules in a fluid bed processor or rapid mixer granulator.
4. Mixing the lubricant and glidant with obtained granules.

In another embodiment, the present invention relates to a provide process for preparation of oral pharmaceutical composition comprising Arsenic trioxide and one or more pharmaceutically acceptable excipients in the form of granules, wherein the said granules are prepared by rotary evaporator; and the process for preparation includes the steps of:
1. Dissolving Arsenic Trioxide with pH adjusting agents in purified water,
2. Preparing the solution of diluent in water separately to get the clear solution.
3. Adding the solution of step 2 to step 1 under stirring and adding glidant to the obtained solution under stirring.
4. Adding the above solution in the rotary evaporator and drying it to obtain the granules.
5. Mixing lubricant, surfactant and glidant with obtained granules.

In another embodiment, the present invention relates to an oral pharmaceutical composition comprising Arsenic trioxide and one or more pharmaceutically acceptable excipients, wherein the said composition is not obtained by lyophilization; and wherein at least 80% of arsenic trioxide in the composition is released within 45 minutes when measured by dissolution test in 900 ml of 0.1 N HCl as dissolution media having paddle with sinker at 100 RPM, or by dissolution test in 250 ml 0.1 N HCl as dissolution media having paddle with sinker at 50 RPM.

In another embodiment, the present invention relates to an oral pharmaceutical composition comprising Arsenic trioxide and one or more pharmaceutically acceptable excipients, wherein the said composition is not obtained by lyophilization; and wherein the said composition can be used for the treatment of acute promyelocytic leukemia and other cancers like acute myeloid leukemia.

### DETAILED DESCRIPTION OF THE INVENTION

The detailed description and the examples provided herein are exemplary and any modification or variation within the scope of the invention will be apparent to a person skilled in the art. Further, unless otherwise defined, all the technical and scientific terms used herein shall bear the meaning as understood by a person who is ordinarily skilled in the art.

In one embodiment, the present invention provides an oral pharmaceutical composition of Arsenic trioxide and one or more pharmaceutically acceptable excipients, wherein the said composition is a capsule, a tablet or granules.

The term “Arsenic trioxide” used within the specification includes Arsenic trioxide or its pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable pro-drugs thereof.

The term “oral pharmaceutical composition” of the present invention means an oral solid dosage form comprising arsenic trioxide in the form of a capsule, a tablet or granules, wherein the said composition is not obtained by lyophilization; and wherein at least 80% of arsenic trioxide in the composition is released within 45 minutes, when measured by dissolution test in 900 ml of 0.1N HCl as dissolution media having paddle with sinker at 100 RPM, or by dissolution test in 250 ml 0.1N HCl as dissolution media having paddle with sinker at 50 RPM.

The term “dissolution profile” as used herein refers to the release of arsenic trioxide from the oral pharmaceutical composition. Hereinafter, the dissolution profile is measured in weight of dissolved arsenic trioxide per initial weight of arsenic trioxide in the dosage form, and it is expressed in weight percentage (% w/w). The dissolution profile of at least 80% of arsenic trioxide release from the oral composition within 45 minutes is obtained, when measured by dissolution test in 900 ml of 0.1N HCl as dissolution media having paddle with sinker at 100 RPM, or by dissolution test in 250 ml 0.1N HCl as dissolution media having paddle with sinker at 50 RPM.

The term “wet granulation process” as used herein refers to a process wherein the granules are formed by the addition of a granulation liquid onto a powder bed under the influence of an impeller (in a rapid mixer granulator) or under the influence of air (in a fluidized bed processor).

The term “spray drying process” as used herein refers to atomization of a liquid feed into very small droplets within hot drying gas leading to flash drying of the droplets into solid particles. The particles are then separated from the drying gas, using a cyclone and/or a filter bag, as spray dried product.

The term “rotary evaporator” as used herein refers to a process for removal of solvents from feed material by evaporation to obtain dried granules.

In another embodiment, the amount of arsenic trioxide in the said oral pharmaceutical composition is in the range from 1 mg to 25 mg, more preferably from 10 mg to 20 mg.

In another embodiment, the present invention relates to an oral pharmaceutical composition of Arsenic trioxide and one or more pharmaceutically acceptable excipients, wherein the said composition is not obtained by lyophilization.

In another embodiment, oral pharmaceutical composition of the present invention comprises pharmaceutically acceptable excipients selected from diluent, binder, lubricant, glidant, surfactant, solvent, pH adjusting agents and optionally disintegrant.

According to the present invention, the diluent may include, but not limited to lactose anhydrous, lactose monohydrate, spray dried lactose, dicalcium phosphate, mannitol (such as Pearlitol and Pearlitol SD), calcium phosphate tribasic, calcium carbonate, calcium sulfate, starch, corn starch, potato starch, wheat starch, pregelatinized starch, microcrystalline cellulose, colloidal silicon dioxide (such as Syloid), silicified microcrystalline cellulose, or mixtures thereof. Preferably the diluent used in the oral pharmaceutical composition of arsenic trioxide is mannitol and silicon dioxide or mixture thereof.

According to present invention, the binder may include, but not limited to guar gum, hydroxyethyl cellulose, hydroxyethyl methyl cellulose, hydroxypropyl cellulose, hydroxypropyl starch, lactose anhydrous, spray-dried lactose, low-substituted hydroxypropyl cellulose, hydroxypropyl methyl cellulose (i.e. HPMC/hypromellose), inulin, and the like, or mixtures thereof. Preferably the binder used in the oral pharmaceutical composition of arsenic trioxide is hypromellose.

According to present invention, the lubricant may include, but not limited to calcium stearate, glyceryl behenate, magnesium stearate, mineral oil light, polyethylene glycol, castor oil, sodium stearyl fumarate, starch, stearic acid, talc, hydrogenated vegetable oil, zinc stearate, sodium benzoate and the like, or mixtures thereof. Preferably the lubricant used in the oral pharmaceutical composition of arsenic trioxide is magnesium stearate.

According to present invention, the glidant may include, but not limited to calcium silicate, magnesium silicate, colloidal silicon dioxide, talc and the like, or mixtures thereof. Preferably the glidant used in the oral pharmaceutical composition of arsenic trioxide is talc.

According to present invention, the surfactant may include, but not limited to cetrimide, cetylpyridinium chloride, docusate sodium, glyceryl monooleate, lauric acid, macrogol 15 hydroxystearate, myristyl alcohol, sodium lauryl sulfate, sorbitan esters and the like, or mixtures thereof. Preferably the surfactant used in the oral pharmaceutical composition of arsenic trioxide is sodium lauryl sulfate.

The solvents for the purpose of the present invention may include purified water. According to present invention, the pH adjusting agents may include, but not limited to sodium hydroxide and hydrochloric acid or mixture thereof.

According to present invention, the disintegrant may include, optionally, but not limited to crospovidone, starch, pregelatinized starch, sodium starch glycolate, ion-exchange resin and the like, or mixtures thereof.

In another embodiment, the amount of diluent concentration can range from about 40% to 90% w/w, the amount of binder concentration can range from about 1% to 12% w/w, the amount of disintegrant concentration can range from about 0% to 10% w/w, the amount of lubricant concentration can range from about 0.5% to 10% w/w, the amount of glidant concentration can range from about 0.5% to 10% w/w and the amount of surfactant concentration can range from about 3% to 8% w/w.

In a preferred embodiment, the present invention relates to an oral pharmaceutical composition comprising Arsenic trioxide and pharmaceutically acceptable excipients selected from mannitol, silicon dioxide, hypromellose, magnesium stearate, talc, sodium lauryl sulfate, sodium hydroxide, hydrochloric acid and water.

In another embodiment, the present invention relates to a process for preparation of oral pharmaceutical composition comprising Arsenic trioxide and one or more pharmaceutically acceptable excipients in the form of granules, wherein the said granules are prepared by wet granulation process, spray drying process or by rotary evaporator.

In another embodiment, the present invention relates to a process for preparation of oral pharmaceutical composition comprising Arsenic trioxide and one or more pharmaceutically acceptable excipients in the form of granules, wherein the said granules are prepared by wet granulation process comprising of fluid bed processor or rapid mixer granulator.

In another embodiment, the present invention relates to a process for preparation of oral pharmaceutical composition of Arsenic trioxide in the form of granules, wherein the granules are prepared by fluid bed processor comprising the steps of:
1. Dissolving Arsenic Trioxide and sodium hydroxide pellets in purified water to get the clear solution.
2. Adjusting the pH to about 8.0 using hydrochloride solution.
3. Preparing the solution of hydroxypropyl methylcellulose (Hypromellose) in water separately to get the clear solution.
4. Adding the solution of step 3 to step 2 under stirring and further adding sodium lauryl sulphate.
5. Adding mannitol into fluid bed processor bowl.
6. Granulating the sifted mannitol of step 5 using solution of step 4.
7. Sifting purified talc and magnesium stearate through suitable sieve and mixing with above granules in the blender.

In another embodiment, the present invention relates to a process for preparation of oral pharmaceutical composition of Arsenic trioxide in the form of granules, wherein the granules are prepared by rotary evaporator comprising the steps of:
1. Dissolving Arsenic Trioxide and sodium hydroxide pellets in purified water to get the clear solution.
2. Adjusting the pH to about 8.0 using hydrochloride solution.
3. Preparing the solution of mannitol in water separately to get the clear solution.
4. Adding the solution of step 3 to step 2 under stirring and adding colloidal silicon dioxide to the resultant solution under stirring.
5. Adding the above solution in the rotary evaporator and drying it to get the granules.
6. Screening the obtained granules using suitable sieve.
7. Sifting purified talc, sodium lauryl sulphate and magnesium stearate through suitable sieve and mixing with granules in the blender.

In another embodiment, the present invention relates to a process for preparation of oral pharmaceutical composition of Arsenic trioxide in the form of granules, wherein the granules are filled into capsules of suitable size or are compressed into tablets.

In another embodiment, the present invention relates to a process for preparation of oral pharmaceutical composition of Arsenic trioxide in the form of granules, wherein the granules are filled into suitable size of capsules.

In another embodiment, the present invention relates to an oral pharmaceutical composition of Arsenic trioxide and one or more pharmaceutically acceptable excipients, wherein the said composition provides enhanced dissolution profile without the need for lyophilization.

In a preferred embodiment, the present invention relates to an oral pharmaceutical composition of Arsenic trioxide, wherein the said composition is a capsule, a tablet or a granule, and wherein at least 80% of arsenic trioxide in the composition is released within 45 minutes when measured by dissolution test in 900 ml of 0.1 N HCl having paddle with sinker at 100 RPM, or by dissolution test in 250 ml 0.1 N HCl having paddle with sinker at 50 RPM.

In a preferred embodiment, the present invention provides an oral pharmaceutical composition comprising Arsenic trioxide and one or more pharmaceutically acceptable excipients, wherein the said composition is not obtained by lyophilization; and wherein the said composition does not comprise more than 0.5% W/W of AS-(V) impurity of Arsenic trioxide, when stored at 40° C./75% RH for three months. The AS-(V) impurity means Arsenic Pentavalent ion.

In order to further illustrate the present invention, the following examples are provided for the purpose of clarity of understanding. However, it is not intended in any way to limit the scope of present invention and it is readily apparent to those of ordinary skill in the art in light of the teachings of this invention that certain changes and modifications may be made thereto without departing from the scope of the invention.

### EXAMPLES

The present invention has been described by way of example only, and it is to be recognized that modifications thereto falling within the scope and spirit of the appended claims, and which would be obvious to a person skilled in the art based upon the disclosure herein, are also considered to be included within the scope of this invention.

### Example-1: Oral Pharmaceutical Composition of Arsenic Trioxide

| Sr. No. | Ingredient | Ingredients (% w/w) |
|---|---|---|
| 1 | Arsenic Trioxide | 1-25 |
| 2 | Diluents | 40-90 |
| 3 | Binders | 1-12 |
| 4 | Disintegrant | 0-10 |
| 5 | Lubricant | 0.5-10 |
| 6 | Glidant | 0.5-10 |
| 7 | Surfactant | 3-8 |
| 8 | Solvent | q.s. |
| 9 | pH adjusting agents | q.s. |

The oral composition can be prepared by wet granulation process (such as fluid bed processor or rapid mixer granulator), spray drying or rotary evaporator.

### Example-2: Oral Pharmaceutical Compositions of Arsenic Trioxide Prepared by Direct Mixing Process and the Dissolution Profile

| Batch No. | |
|---|---|
| Batch-1 | Batch-2 |

| Contents | mg/cap | % w/w | mg/cap | % w/w |
|---|---|---|---|---|
| Arsenic Trioxide | 15.00 | 10.00 | 15.00 | 10.00 |
| Mannitol | 132.00 | 88.00 | 124.50 | 83.00 |
| Sodium Lauryl | — | — | 7.50 | 7.50 |
| Sulphate | | | | |
| Purified Talc | 1.50 | 1.00 | 1.50 | 1.00 |
| Magnesium Stearate | 1.50 | 1.00 | 1.50 | 1.00 |
| Total | 150.00 | 100.00 | 150.00 | 100.00 |

| | | | | |
|---|---|---|---|---|
| Timings | % Release | % RSD | % Release | % RSD |

Dissolution (0.1N HCl, 900 ml, Paddle with Sinker at 100 RPM)

| | | | | |
|---|---|---|---|---|
| 10 min | 2 | 4.92 | 3 | 7.6 |
| 15 min | 2 | 10.33 | 3 | 6.47 |
| 20 min | 3 | 8.43 | 4 | 5.34 |
| 30 min | 3 | 9.11 | 4 | 8.28 |
| 45 min | 4 | 10.08 | 6 | 4.04 |
| 60 min | 5 | 5.57 | 7 | 6.24 |

Dissolution (0.1N HCl, 250 ml, Paddle with Sinker at 50 RPM)

| | | | | |
|---|---|---|---|---|
| 10 min | 0 | 0 | 0 | 0 |
| 15 min | 0 | 0 | 0 | 0 |
| 20 min | 0 | 0 | 0 | 0 |
| 30 min | 1 | 6.33 | 1 | 8.94 |
| 45 min | 1 | 4.08 | 1 | 18.71 |
| 60 min | 1 | 4.08 | 1 | 13.78 |

% RSD = Relative standard deviation.

Manufacturing Process:
1. Sifting Arsenic Trioxide, sodium lauryl sulphate and Mannitol through suitable sieve and mixing in blender,
2. Sifting purified talc through suitable sieve and mixing with blend of step-1,
3. Sifting magnesium stearate through suitable sieve and mixing with above blend in blender,
4. Filling the lubricated blend in capsules or compressed into tablet.

The dissolution studies of the above-mentioned oral composition of Arsenic trioxide were carried out in: (a) 900 ml 0.1 N HCl as dissolution media in paddle with sinker at 100 RPM, and (b) 250 ml 0.1 N HCl as dissolution media in paddle with sinker at 50 RPM. The dissolution data shows that the release of Arsenic trioxide from the oral composition prepared by direct mixing process is very slow and incomplete.

### Example-3: Oral Pharmaceutical Compositions of Arsenic Trioxide Prepared by Wet Granulation Method and the Dissolution Profile

| | Batch No. | | | |
|---|---|---|---|---|
| | Batch 1 | | Batch 2 | |
| Contents | mg/cap | % w/w | mg/cap | % w/w |
| Arsenic Trioxide | 15.00 | 10.00 | 15.00 | 10.00 |
| Sodium Hydroxide | 15.00 | 10.00 | 15.00 | 10.00 |
| Purified Water | q.s. | q.s. | q.s. | q.s. |
| HCl | q.s. | q.s. | q.s. | q.s. |
| Hypromellose E5 | 12.00 | 8.00 | 12.00 | 8.00 |
| Purified Water | q.s. | q.s. | q.s. | q.s. |
| Mannitol | 105.00 | 70.00 | 97.50 | 65.00 |
| Sodium Lauryl Sulphate | — | — | 7.50 | 5.00 |
| Purified Talc | 1.50 | 1.00 | 1.50 | 1.00 |
| Magnesium Stearate | 1.50 | 1.00 | 1.50 | 1.00 |
| | 150 | 100 | 150 | 100 |

| | | | | |
|---|---|---|---|---|
| Timings | % Release | % RSD | % Release | % RSD |

Dissolution (0.1N HCl, 900 ml, Paddle with Sinker at 100 RPM)

| | | | | | |
|---|---|---|---|---|---|
| 10 min | 71 (54-87) | 19.54 | 94 | (89-103) | 5.32 |
| 15 min | 87 (76-94) | 9.24 | 98 | (91-107) | 5.79 |
| 20 min | 91 (87-94) | 2.9 | 98 | (90-109) | 6.83 |
| 30 min | 93 (91-95) | 1.33 | 98 | (89-110) | 7.47 |
| 45 min | 94 (93-96) | 1.29 | 98 | (91-109) | 7.1 |
| 60 min | 95 (93-97) | 1.33 | 99 | (91-111) | 7.44 |

Dissolution (0.1N HCl, 250 ml, Paddle with Sinker at 50 RPM)

| | | | | | |
|---|---|---|---|---|---|
| 10 min | 27 (23-30) | 9.53 | 61 | (51-70) | 11.18 |
| 15 min | 54 (49-59) | 6.39 | 75 | (65-81) | 7.51 |
| 20 min | 72 (65-78) | 6.13 | 77 | (64-83) | 8.96 |
| 30 min | 86 (85-87) | 1.04 | 79 | (68-84) | 7.78 |
| 45 min | 87 (85-89) | 1.72 | 82 | (70-88) | 8.23 |
| 60 min | 88 (84-90) | 2.25 | 82 | (70-87) | 7.54 |

% RSD = Relative standard deviation.

Manufacturing Process:
1. Dissolving Arsenic Trioxide and sodium hydroxide pellets in purified water to get the clear solution,
2. Adjusting the pH to about 8.0 using hydrochloride solution,
3. Adding hypromellose and sodium lauryl sulphate into the obtained solution,
4. Adding mannitol into a fluid bed processor or a rapid mixer granulator,
5. Granulating the sifted mannitol to obtain granules,
6. Sifting purified talc and magnesium stearate through suitable sieve and mixing with above granules for in the blender,
7. Optionally granules are filled into capsules or compressed into tablet.

The dissolution studies of the above-mentioned oral composition of Arsenic trioxide showed that the release of Arsenic trioxide from the oral composition prepared by fluid bed processor was above 80% within 45 minutes measured by dissolution test in 900 ml, 0.1 N HCl, USP apparatus (paddle with sinker) at 100 RPM, or by dissolution test in 250 ml, 0.1 N HCl, USP apparatus (paddle with sinker) at 50 RPM.

### Example-4: Oral Pharmaceutical Compositions of Arsenic Trioxide Prepared by Rotary Evaporator Method and the Dissolution Profile

| | Batch No. | | | |
|---|---|---|---|---|
| | Batch 1 | | Batch 2 | |
| Composition | mg/cap | % w/w | mg/cap | % w/w |
| Arsenic Trioxide | 15.00 | 10.03 | 15.00 | 9.55 |
| Sodium Hydroxide | 15.00 | 10.03 | 15.00 | 9.55 |
| Purified Water | q.s. | q.s. | q.s. | q.s. |
| HCl | q.s. | q.s. | q.s. | q.s. |
| Mannitol | 66.50 | 44.48 | 66.50 | 42.36 |
| Purified Water | q.s. | q.s. | q.s. | q.s. |
| Colloidal silicon dioxide | 40.00 | 26.76 | 40.00 | 25.48 |
| Mannitol | 10.00 | 6.69 | 10.00 | 6.37 |
| Sodium Lauryl Sulphate | — | — | 7.50 | 4.78 |
| Purified Talc | 1.50 | 1.00 | 1.50 | 0.96 |
| Magnesium Stearate | 1.50 | 1.00 | 1.50 | 0.96 |
| | 149.5 | 100 | 157 | 100 |

| | | | | |
|---|---|---|---|---|
| Timings | % Release | % RSD | % Release | % RSD |

Dissolution (0.1N HCl, 900 ml, Paddle with Sinker at 100 RPM)

| | | | | | | |
|---|---|---|---|---|---|---|
| 10 min | 86 | (74-94) | 8.76 | 98 | (96-100) | 1.72 |
| 15 min | 93 | (91-97) | 2.9 | 97 | (96-99) | 1.33 |
| 20 min | 95 | (92-97) | 2.22 | 97 | (95-100) | 1.5 |
| 30 min | 97 | (94-100) | 2.64 | 98 | (96-100) | 1.61 |
| 45 min | 98 | (95-101) | 2.57 | 98 | (96-101) | 1.75 |
| 60 min | 98 | (96-101) | 2.05 | 98 | (96-101) | 1.85 |

Dissolution (0.1N HCl, 250 ml, Paddle with Sinker at 50 RPM)

| | | | | | | |
|---|---|---|---|---|---|---|
| 10 min | 85 | (77-91) | 6.18 | 96 | (93-99) | 2.55 |
| 15 min | 92 | (89-95) | 2.67 | 101 | (101-102) | 0.51 |
| 20 min | 94 | (91-98) | 2.33 | 101 | (101-101) | 0.31 |
| 30 min | 96 | (94-99) | 1.82 | 101 | (101-102) | 0.28 |
| 45 min | 97 | (95-101) | 1.9 | 102 | (101-102) | 0.32 |
| 60 min | 98 | (96-101) | 1.92 | 102 | (102-103) | 0.42 |

% RSD = Relative standard deviation.

Manufacturing Process:
1. Dissolving Arsenic Trioxide and sodium hydroxide pellets in purified water to get the clear solution,
2. Adjusting the pH to about 8.0 using hydrochloride solution,
3. Adding mannitol and Colloidal silicon dioxide to the obtained solution,
4. Adding the above solution in the rotary evaporator and drying it to obtain the granules,
5. Screening the obtained granules using suitable sieve,
6. Sifting purified talc, sodium lauryl sulphate and magnesium stearate through suitable sieve and mixing with granules in the blender,
7. Optionally granules are filled into capsules or compressed into tablet.

The dissolution studies of the above-mentioned Oral composition of Arsenic trioxide showed that the release of Arsenic trioxide from the oral composition prepared by rotary evaporator was above 80% within 45 minutes measured by dissolution test in 900 ml, 0.1 N HCl, paddle with sinker at 100 RPM, or by dissolution test in 250 ml, 0.1 N HCl, paddle with sinker at 50 RPM.

### Example-5: Oral Pharmaceutical Composition of Arsenic Trioxide by Rotary Evaporator Method and the Dissolution Profile

Batch No.
Batch-3

| Composition | mg/cap | % w/w |
|---|---|---|
| Arsenic Trioxide | 15.00 | 9.38 |
| Sodium Hydroxide | 15.00 | 9.38 |
| Purified Water | q.s. | — |
| HCl | q.s. | — |
| Mannitol | 63.30 | 39.56 |
| Purified Water | q.s. | — |
| Colloidal silicon dioxide | 40.00 | 25.00 |
| Mannitol | 16.00 | 10.00 |
| Sodium Lauryl Sulphate | 7.50 | 4.69 |
| Purified Talc | 1.60 | 1.00 |
| Magnesium Stearate | 1.60 | 1.00 |
| Total | 160.00 | 100.00 |

| | | |
|---|---|---|
| Timings | % Release | % RSD |

Dissolution (0.1N HCl, 900 ml, Paddle with Sinker at 100 RPM)

| | | | |
|---|---|---|---|
| 10 min | 92 | (66-109) | 16.08 |
| 15 min | 100 | (93-110) | 5.51 |
| 20 min | 101 | (98-110) | 4.22 |
| 30 min | 101 | (98-109) | 4.38 |
| 45 min | 101 | (97-109) | 4.37 |
| 60 min | 101 | (97-110) | 4.78 |

Dissolution (0.1N HCl, 250 ml, Paddle with Sinker at 50 RPM)

| | | | |
|---|---|---|---|
| 10 min | 30 | (9-43) | 44.25 |
| 15 min | 65 | (56-73) | 10.72 |
| 20 min | 88 | (73-104) | 15.58 |
| 30 min | 99 | (91-105) | 5.70 |
| 45 min | 99 | (94-106) | 5.59 |
| 60 min | 101 | (95-106) | 5.30 |

% RSD = Relative standard deviation.

The composition of Example-5 was prepared by the process described in the Example-4. The dissolution studies of the Oral composition of Arsenic trioxide showed that the release of Arsenic trioxide from the oral composition prepared by rotary evaporator was above 80% within 45 minutes measured by dissolution test in 900 ml, 0.1 N HCl, paddle with sinker at 100 RPM, or by dissolution test in 250 ml, 0.1 N HCl, paddle with sinker at 50 RPM.

### Example-6: Stability Studies of Oral Composition of Arsenic Trioxide Prepared by Rotary Evaporator Method

| | Batch Details/Remark | | |
|---|---|---|---|
| | Batch-3 by Rotary Evaporator | | |
| | (Granules filled in Capsule) | | |
| | Strength | | |
| | 15 mg | | |
| | Condition | | |
| | | 40° C./75% RH - | 40° C./75% RH - |
| | Initial | 2 Months | 3 Months |
| Pack | — | ALU ALU | ALU ALU |
| | | BLISTER | BLISTER |
| Description | Complies | Complies | Complies |
| Water Content (%) | 3.90% | 3.80% | 3.20% |
| Dissolution | 101% | 97% | 100% |
| AS-(V) Impurity (%) | 0.088% | 0.356% | 0.418% |
| Assay (%) | 100.40% | 100.20% | 97.60% |
| Average weight of | 202.2 mg | 202.9 mg | 198.3 mg |
| filled capsule (mg) | | | |
| Average Net | 161.0 mg | 162.2 mg | 157.3 mg |
| content (mg) | | | |

It was observed that the drug content (lubricated blend) filled into capsules shows improved moisture protection (i.e. controlled water content) and better impurity profile. Such oral pharmaceutical compositions comprising Arsenic trioxide does not comprise more than 0.5% W/W of AS-(V) impurity of Arsenic trioxide, when stored at 40° C./75% for three months.

The detailed description and the example provided herein are exemplary and any modification or variation within the scope of the invention will be apparent to a person skilled in the art. Further, unless otherwise defined, all the technical and scientific terms used herein shall bear the meaning as understood by a person who is ordinarily skilled in the art.

1. An oral pharmaceutical composition comprising Arsenic trioxide and one or more pharmaceutically acceptable excipients, wherein the said composition is not obtained by lyophilization; and wherein the said composition is in the form of a capsule, a tablet or granules.

2. The oral pharmaceutical composition comprising Arsenic trioxide according to claim 1, wherein the amount of arsenic trioxide in the said composition is from about 1 mg to 25 mg.

3. The oral pharmaceutical composition comprising Arsenic trioxide and one or more pharmaceutically acceptable excipients according to claim 1, wherein the composition comprises pharmaceutically acceptable excipients selected from diluent, binder, lubricant, glidant, surfactant, solvent, pH adjusting agents and optionally disintegrant.

4. The oral pharmaceutical composition comprising Arsenic trioxide and one or more pharmaceutically acceptable excipients according to claim 1, wherein the said composition is prepared by wet granulation process or by rotary evaporator.

5. A process for preparation of an oral pharmaceutical composition according to claim 1, wherein the said composition is prepared by wet granulation process; and the said process for preparation includes the steps of:
a) Dissolving Arsenic Trioxide with pH adjusting agents in purified water,
b) Adding binder and surfactant to the solution obtained in step a),
c) Granulating the diluent with the solution obtained in step b), to obtain the granules in a fluid bed processor or a rapid mixer granulator,
d) Mixing the lubricant and glidant with obtained granules,
e) Optionally granules are filled into capsules or compressed into tablet.

6. A process for preparation of an oral pharmaceutical composition according to claim 1, wherein the said composition is prepared by rotary evaporator process; and the said process for preparation includes the steps of:
a) Dissolving Arsenic Trioxide with pH adjusting agents in purified water,
b) Adding the diluent to the obtained solution,
c) Adding the above solution in the rotary evaporator and drying it to obtain the granules,
d) Mixing lubricant, surfactant and glidant with obtained granules,
e) Optionally granules are filled into capsules or compressed into tablet.

7. An oral pharmaceutical composition comprising Arsenic trioxide and one or more pharmaceutically acceptable excipients, wherein the said composition is not obtained by lyophilization; and wherein at least 80% of arsenic trioxide in the composition is released within 45 minutes when measured by dissolution test in 900 ml of 0.1N HCl having paddle with sinker at 100 RPM, or by dissolution test in 250 ml of 0.1N HCl having paddle with sinker at 50 RPM.

8. An oral pharmaceutical composition comprising Arsenic trioxide and one or more pharmaceutically acceptable excipients, wherein the said composition is not obtained by lyophilization; and wherein the said composition does not comprise more than 0.5% W/W of AS-(V) impurity of Arsenic trioxide, when stored at 40° C./75% RH for three months.

9. The oral pharmaceutical composition comprising Arsenic trioxide and one or more pharmaceutically acceptable excipients according to claim 1, wherein the said composition can be used for the treatment of acute promyelocytic leukemia and acute myeloid leukemia.
